(19) United States
(12) Patent Application Publication
(10) Pub. No.: US 2008/0312327 A1
(43) Pub. Date: Dec. 18, 2008

(54) Organic Compositions

(75) Inventors: Ralph Edward Rypkema, Lodi, NJ (US); Haoming Yuan, Montvale, NJ (US)

Correspondence Address: NORRIS, MCLAUGHLIN & MARCUS, 875 THIRD AVE, 18TH FLOOR, NEW YORK, NY, 10022, US

(73) Assignee: RECKITT BENCKISER INC., Parsippany, NJ (US)

(21) Appl. No.: 11/574,323

(22) PCT Filed: Aug. 26, 2005
(86) PCT No.: PCT/GB05/03340

§ 371 Date: Jul. 2, 2007

(30) Foreign Application Priority Data
Sep. 11, 2004 (GB) 0420194.3

(51) Int. Cl. C11D 3/48; C11D 3/20; A61K 31/045; A61K 31/192; A61P 31/00

(52) U.S. Cl. 514/570; 510/131; 510/119; 514/739; 514/737

(57) ABSTRACT

Provided is an improved liquid composition for the treatment of surfaces, preferably the skin and/or hair in order to provide a cleaning and/or sanitizing effect thereto, which necessarily comprises: one or more detersive surfactants selected from anionic, nonionic, cationic, and amphoteric surfactants; a first biocidal constituent(s) which provide a biocidal effect in conjunction with at least one or more second biocidal constituent(s) selected from benzoic acid, benzoic acid derivatives and salts thereof or farnesol, one or more pH adjusting constituents, optionally one or more organic solvents, hydrotropes, and further additive constituents, and water. In preferred embodiments the hydrotrope and/or organic solvents are desirably excluded from the compositions, and the composition is thickened. The compositions are particularly useful as a hand wash composition, body wash or shampoo.

The present invention generally relates to improved surface treatment compositions and methods for treating surfaces. More specifically the present invention relates to improved liquid compositions for the treatment of the skin and or hair in order to provide a cleaning and/or sanitizing effect thereto, and methods for providing such a cleaning and/or sanitizing effect.

The prior art has suggested many liquid compositions which contain one or more surfactants for the cleaning surfaces, particularly skin surfaces and hair in order to release dirt and/or microorganisms from these surfaces. Such compositions are widely known and used and are often referred to as “liquid soaps”, “liquid handsoaps”, “shampoos”, “shower foam”, “body wash” and “body soaps”. Such compositions are often also available in the form of “light duty” liquid dishwashing detergent compositions although such are primarily marketed for the hand washing of dishes and other cooking and or eating utensils. Of importance, but less widely knows or available are liquid compositions containing one or more surfactants for providing a primary cleaning benefit, in conjunction with one or more biocidal agents which provide a degree of sanitization to the treated surfaces. The incorporation of such biocidal agents is a technical problem which requires care in formulation in order to ensure that the beneficial benefits of the one or more surfactants, and the one or more biocidal agents are not unduly compromised when combined into a liquid composition. Ideally such a liquid composition should, in addition to providing good cleaning and/or disinfecting efficacy to treated compositions should also exhibit good storage stability, and pose reduced likelihood of irritation to the user of the product, particularly when such a product is to be applied directly to the skin and or hair of a user.

For example, PCT/GB02/02441 describes bacteridical liquid detergent compositions which necessarily include: water, one or more surfactants, one or more specific non-ionic, non-phenolic antimicrobial agents, and either an aromatic sulphonate hydrotrope or a water soluble organic solvent but preferably both, and optionally further minor constituents. Therein is recited that the presence of the aromatic sulphonate hydrotrope is required as it is believed that it provides a synergistic disinfecting benefit when combined with the specific non-ionic, non-phenolic antimicrobial agents.

In PCT/GB2003/004381 are described liquid detergent compositions which necessarily include water, at least one surfactant, and at least two different organic acids or salts thereof which latter constituents provide at least 0.5% (v/v), preferably at least 1% (v/v) of the compositions. Preferably the at least two different organic acids or salts thereof are saturated aliphatic carboxylic acids and/or carboxylates, and especially are ones having two or more carboxylic acid or carboxylate groups. The compositions may further include a biocidal agent, however according to certain preferred embodiments a traditional biocidal agent is omitted from the compositions as it is believed that the at least two different organic acids or salts thereof provide both a buffering effect and an biocidal effect as well.

In PCT/EP98/02565 are recited dishwashing detergent compositions which provide an antibacterial effect which compositions comprise water, one or more detersive surfactants and preferably benzoic acid or salts thereof which are recited to be useful in providing a biocidal effect.

The prior art has suggested a number of compositions which are intended to address this technical need, however, these compositions are not without shortcomings and thus also exhibit a real need in the art for further improved compositions for the treatment of hard surface, especially improved compositions for the treatment of the skin and or hair which provide a cleaning and disinfecting benefit thereto.

In one aspect of the present invention there is provided a liquid composition for the treatment of the skin and/or hair in order to provide a cleaning and/or sanitizing effect thereto, which necessarily comprises:

1-35% wt. of one or more detersive surfactants selected from anionic, nonionic, cationic, and amphoteric surfactants;

0.01-5% wt. of one or more first biocidal constituent(s) which provide a biocidal effect in conjunction with at least one or more second biocidal constituent(s) selected from selected from benzoic acid, benzoic acid derivatives and/or salts thereof, and farnesol;

0.01-7% wt. of one or more pH adjusting constituents;

0-10% wt. of one or more organic solvents;

0-10% wt. of one or more hydrotropes;

0-50% wt. of one or more optional constituents including but not limited to: thickeners, viscosity modifiers, coloring agents, opacifying agents, dyes, fragrances, perfumes, preservatives, chelating agents; and

to 100% wt. water.

According to a second aspect of the invention there is provided a liquid composition described according to the first aspect of the invention which further includes necessarily includes a thickener constituent. Preferably the thickener constituent is present in a sufficient thickening-effective amount such that viscosity of the liquid composition is at least about 2,000 cps, preferably at least about 5,000 cps.

According to a third aspect of the invention there is provided a liquid composition described according to the first or second aspects of the invention wherein hydrotropes, particularly hydrotropes based on sulfonated compounds are excluded from the compositions.

According to the fourth aspect of the invention there is provided a liquid composition described according to the first, second or third aspects of the invention wherein organic solvents are excluded from the inventive compositions.

According to a further aspect of the invention, there are provided methods for providing such a cleaning and/or sanitizing effect which contemplate the application of one or more of the liquid compositions described herein to a surface, particularly skin and/or hair in order to impart a cleaning and/or sanitizing effect thereto.

The inventive compositions include 1-35% wt. of one or more detersive surfactants selected from anionic, nonionic, cationic, amphoteric or zwitterionic surfactants.

Exemplary anionic surfactants include but are not limited to: alkali metal salts, ammonium salts, amine salts, aminoalcohol salts or the magnesium salts of one or more of the following compounds: alkyl sulfates, alkyl ether sulfates, alkylamidoether sulfates, alkylaryl polyether sulfates, monoglyceride sulfates, alkylsulfonates, alkylamide sulfonates, alkylarylsulfonates, olefinsulfonates, paraffin sulfonates, alkyl sulfosuccinates, alkyl ether sulfosuccinates, alkylamide sulfosuccinates, alkyl sulfosuccinamate, alkyl sulfoacetates, alkyl phosphates, alkyl ether phosphates, acyl sarconsinates, acyl isethionates, and N-acyl taurates. Generally, the alkyl or acyl radical in these various compounds comprise a carbon chain containing 12 to 20 carbon atoms.

Further exemplary anionic surface active agents which may be used include fatty acid salts, including salts of oleic, ricinoleic, palmitic, and stearic acids; copra oils or hydrogenated copra oil acid, and acyl lactylates whose acyl radical contains 8 to 20 carbon atoms.

Exemplarly useful anionic surface active agents, also known as anionic surfactants include the water-soluble salts, particularly the alkali metal, ammonium and alkylolammonium (e.g., monoethanolammonium or triethanolammonium) salts, of organic sulfuric reaction products having in their molecular structure an alkyl group containing from about 10 to about 20 carbon atoms and a sulfonic acid or sulfuric acid ester group. (Included in the term “alkyl” is the alkyl portion of aryl groups.) Examples of this group of synthetic surfactants are the alkyl sulfates, especially those obtained by sulfating the higher alcohols (C₈-C₁₈ carbon atoms) such as those produced by reducing the glycerides of tallow or coconut oil; and the alkylbenzene sulfonates in which the alkyl group contains from about 9 to about 15 carbon atoms, in straight chain or branched chain. Especially valuable are linear straight chain alkylbenzene sulfonates in which the average number of carbon atoms in the alkyl group is from about 11 to 14.

Other anionic surfactants herein are the water soluble salts of: paraffin sulfonates containing from about 8 to about 24 (preferably about 12 to 18) carbon atoms; alkyl glyceryl ether sulfonates, especially those ethers of C₈-C₁₈ alcohols (e.g., those derived from tallow and coconut oil); alkyl phenol ethylene oxide ether sulfates containing from about 1 to about 4 units of ethylene oxide per molecule and from about 8 to about 12 carbon atoms in the alkyl group; and alkyl ethylene oxide ether sulfates containing about 1 to about 4 units of ethylene oxide per molecule and from about 10 to about 20 carbon atoms in the alkyl group.

Other useful anionic surfactants herein include the water soluble salts of esters of α-sulfonated fatty acids containing from about 0 to 20 carbon atoms in the fatty acid group and from about 1 to 10 carbon atoms in the ester group; water soluble salts of 2-acyloxy-alkane-1-sulfonic acids containing from about 2 to 9 carbon atoms in the acyl group and from about 9 to about 23 carbon atoms in the alkane moiety; water-soluble salts of olefin sulfonates containing from about 12 to 24 carbon atoms; and β-alkyloxy alkane sulfonates containing from about 1 to 3 carbon atoms in the alkyl group and from about 8 to 20 carbon atoms in the alkane moiety.

Particularly useful are alkyl sulfate anionic surfactants according to the formula

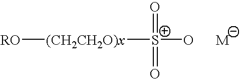

wherein R is an straight chain or branched alkyl chain having from about 8 to about 18 carbon atoms, saturated or unsaturated, and the longest linear portion of the alkyl chain is 15 carbon atoms or less on the average, M is a cation which makes the compound water soluble especially an alkali metal such as sodium, or is ammonium or substituted ammonium cation, and x is from 0 to about 4. Of these, most preferred are the non-ethoxylated C₁₂-C₁₅ primary and secondary alkyl sulfates. Exemplary commercially available alkyl sulfates include one or more of those available under the tradename RHODAPON® from Rhône-Poulenc Co. (Cherry Hill, N.J.) as well as STEPANOL® from Stepan Chemical Co. (Northfield, Ill.).

Also particularly useful are alkyl sulfonate anionic surfactants according to the formula:

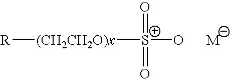

wherein R is an straight chain or branched alkyl chain having from about 8 to about 18 carbon atoms, saturated or unsaturated, and the longest linear portion of the alkyl chain is 15 carbon atoms or less on the average, M is a cation which makes the compound water soluble especially an alkali metal such as sodium, or is ammonium or substituted ammonium cation, and x is from 0 to about 4. Of these, most preferred are the C12-15 primary and secondary alkyl sulfates. Exemplary, commercially available alkane sulfonate surfactants include one or more of those available under the tradename HOSTAPUR®.

Other anionic surfactants not particularly enumerated here may also find use in conjunction with the compositions of the present invention.

Practically any hydrophobic compound having a carboxy, hydroxy, amido, or amino group with a free hydrogen attached to the nitrogen can be condensed with ethylene oxide or with the polyhydration product thereof, polyethylene glycol, to form a water soluble nonionic surfactant compound. Further, the length of the polyethylenoxy hydrophobic and hydrophilic elements may various. Exemplary nonionic compounds include the polyoxyethylene ethers of alkyl aromatic hydroxy compounds, e.g., alkylated polyoxyethylene phenols, polyoxyethylene ethers of long chain aliphatic alcohols, the polyoxyethylene ethers of hydrophobic propylene oxide polymers, and the higher alkyl amine oxides.

A useful class of nonionic surfactants include alkoxy block copolymers which include nonionic surfactants in which the major portion of the molecule is made up of block polymeric C₂-C₄ alkylene oxides. Such nonionic surfactants, while preferably built up from an alkylene oxide chain starting group, and can have as a starting nucleus almost any active hydrogen containing group including, without limitation, amides, phenols, thiols and secondary alcohols.

One group of such useful nonionic surfactants containing the characteristic alkylene oxide blocks are those which may be generally represented by the formula (A):

HO-(EO)ₓ(PO)_{y}(EO)_{z}-H (A)

where

EO represents ethylene oxide,

PO represents propylene oxide,

y equals at least 15,

(EO)_{x+z} equals 20 to 80% of the total weight of said compounds, and,

the total molecular weight is preferably in the range of about 2000 to 15,000.

Another group of nonionic surfactants appropriate for use in the new compositions can be represented by the formula (B):

R-(EO,PO)ₐ(EO,PO)_{b}-H (B)

wherein R is an alkyl, aryl or aralkyl group, where the R group contains 1 to 20 carbon atoms, the weight percent of EO is within the range of 0 to 45% in one of the blocks a, b, and within the range of 60 to 100% in the other of the blocks a, b, and the total number of moles of combined EO and PO is in the range of 6 to 125 moles, with 1 to 50 moles in the PO rich block and 5 to 100 moles in the EO rich block.

Further nonionic surfactants which in general are encompassed by Formula B include butoxy derivatives of propylene oxide/ethylene oxide block polymers having molecular weights within the range of about 2000-5000. Still further useful nonionic surfactants containing polymeric butoxy (BO) groups can be represented by formula (C) as follows:

RO-(BO)ₙ(EO)ₓ-H (C)

wherein

R is an alkyl group containing 1 to 20 carbon atoms,

n is about 5-15 and x is about 5-15.

Also useful as the nonionic block copolymer surfactants, which also include polymeric butoxy groups, are those which may be represented by the following formula (D) as follows:

HO-(EO)ₓ(BO)ₙ(EO)_{y}-H (D)

wherein

n is about 5-15, preferably about 15,

x is about 5-15, preferably about 15, and

y is about 5-15, preferably about 15.

Still further useful nonionic block copolymer surfactants include ethoxylated derivatives of propoxylated ethylene diamine, which may be represented by the following formula:

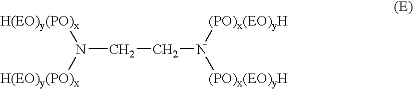

where

(EO) represents ethoxy,

(PO) represents propoxy,

the amount of (PO)ₓ is such as to provide a molecular weight prior to ethoxylation of about 300 to 7500, and the amount of (EO)_{y} is such as to provide about 20% to 90% of the total weight of said compound.

Further useful nonionic surfactant compounds include amine oxides. Exemplary amine oxides include:

(1) Alkyl di (lower alkyl) amine oxides in which the alkyl group has about 10-20, and preferably 12-16 carbon atoms, and can be straight or branched chain, saturated or unsaturated. The lower alkyl groups include between 1 and 7 carbon atoms. Examples include lauryl, dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide;

(2) Alkyl di (hydroxy lower alkyl) amine oxides in which the alkyl group has about 10-20, and preferably 12-16 carbon atoms, and can be straight or branched chain, saturated or unsaturated. Examples are bis(2-hydroxyethyl) cocoamine oxide, bis(2-hydroxyethyl) tallowamine oxide; and bis(2-hydroxyethyl) stearylamine oxide;

(3) Alkylamidopropyl di(lower alkyl) amine oxides in which the alkyl group has about 10-20, and preferably 12-16 carbon atoms, and can be straight or branched chain, saturated or unsaturated. Examples are cocoamidopropyl dimethyl amine oxide and tallowamidopropyl dimethyl amine oxide; and

(4) Alkylmorpholine oxides in which the alkyl group has about 10-20, and preferably 12-16 carbon atoms, and can be straight or branched chain, saturated or unsaturated.

Further exemplary and preferred nonionic surfactants are ethoxylated alcohols and ethoxylated phenols. Such nonionic surfactants are well known and may be formed by condensation of an alkyl phenol, an aliphatic alcohol, or mixtures thereof, with sufficient ethylene oxide to produce a compound having a polyoxyethylene. Preferably the number of ethylene oxide units are present in an amount sufficient to insure solubility of the compound in an aqueous composition of this invention or in any dilution thereof. More preferably the ethoxylated alcohols and phenols are produced by condensation of about 4-16 (more preferably 8-13), moles of ethylene oxide with 1 mole of the parent compound (i.e. alkyl phenol or aliphatic alcohol). As known to those skilled in the art, the number of moles of ethylene oxide which are condensed with one mole of parent compound depends upon the molecular weight of the hydrophobic portion of the condensation product. The parent compounds that may be combined with the ethylene oxide may include one or more of the following:

(1) an alkyl phenol having about 1-15, and preferably 7-10, carbon atoms (saturated or unsaturated) in the alkyl group [including phenol, methyl phenol (cresol), ethyl phenol, hexyl phenol, octyl phenol, dicylphenol, nonylphenol, dodecylphenol, and the like]; and

(2) a primary, tertiary, or secondary aliphatic alcohol having about 10-20, and preferably 11-15, carbon atoms, (including decyl alcohol, dodecyl alcohol, tridecyl alcohol, hexadecyl alcohol, octadecyl alcohol, and the like).

For the alcohol ethoxylates, the alkyl chain of the aliphatic alcohols can be linear or branched, primary or secondary, and generally contains from about 8 to about 22 carbon atoms. The alkyl chain can be saturated or unsaturated. The alcohol ethoxylates can have a narrow (“narrow range ethoxylates”) or a broad (“broad range ethoxylates”) homolog distribution of the ethylene oxide. Examples of commercially available nonionic surfactants of this type are available under the tradenames Tergitol®, Genapol®, and Neodol®.

Further exemplary useful nonionic surfactants include alkanolamides including monoethanolamides and diethanolamides, particularly fatty monoalkanolamides and fatty dialkanolamides. Particularly useful includes C₈-C₂₄ alkyl di(C₂-C₃ alkanol amides), as represented by the following formula:

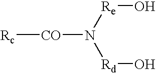

wherein R_{c} is a branched or straight chain C₈-C₂₄ alkyl radical, preferably a C₁₀-C₁₆ alkyl radical and more preferably a C₁₂-C₁₄ alkyl radical, and R_{d} and Rₑ are a C₁-C₄ alkyl radical, preferably an ethyl radical; R_{d} and Rₑ may be the same or different. Particularly useful alkanolamides according to the foregoing structure include cocoamide diethanol amide, and soyamide diethanolamide.

Other nonionic surfactants not particularly enumerated here may also find use in conjunction with the compositions of the present invention.

Exemplary cationic surfactants include known art cationic surfactants. Preferably however the cationic surfactants used in the inventive compositions are compounds which provide a broad antibacterial or sanitizing function. Any cationic surfactant which satisfies these requirements may be used and are considered to be within the scope of the present invention, and mixtures of two or more cationic surface active agents, viz., cationic surfactants may also be used. Cationic surfactants are well known, and useful cationic surfactants may be one or more of those described for example in McCutcheon's Functional Materials, Vol. 2, 1998; Kirk-Othmer, Encyclopedia of Chemical Technology, 4th Ed., Vol. 23, pp. 481-541 (1997), the contents of which are herein incorporated by reference. These are also described in the respective product specifications and literature available from the suppliers of these cationic surfactants.

Examples of preferred cationic surfactant compositions useful in the practice of the instant invention are those which provide a germicidal effect to the concentrate compositions, and especially preferred are quaternary ammonium compounds and salts thereof, which may be characterized by the general structural formula:

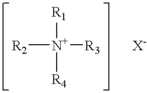

where at least one of R₁, R₂, R₃ and R₄ is a alkyl, aryl or alkylaryl substituent of from 6 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons usually containing no more than 12 carbon atoms. The substituents R₁, R₂, R₃ and R₄ may be straight-chained or may be branched, but are preferably straight-chained, and may include one or more amide, ether or ester linkages. The counterion X may be any salt-forming anion which permits water solubility of the quaternary ammonium complex.

Exemplary quaternary ammonium salts within the above description include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide, and the like. Other suitable types of quaternary ammonium salts include those in which the molecule contains either amide, ether or ester linkages such as octyl phenoxy ethoxy ethyl dimethyl benzyl ammonium chloride, N-(laurylcocoaminoformylmethyl)-pyridinium chloride, and the like. Other very effective types of quaternary ammonium compounds which are useful as germicides include those in which the hydrophobic radical is characterized by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride, chlorinated dodecylbenzyltrimethyl ammonium chloride, and the like.

Preferred quaternary ammonium compounds which act as germicides and which are be found useful in the practice of the present invention include those which have the structural formula:

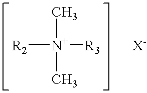

wherein R₂ and R₃ are the same or different C₈-C₁₂alkyl, or R₂ is C₁₂₋₁₆alkyl, C₈₋₁₈alkylethoxy, C₈₋₁₈alkylphenolethoxy and R₃ is benzyl, and X is a halide, for example chloride, bromide or iodide, or is a methosulfate anion. The alkyl groups recited in R₂ and R₃ may be straight-chained or branched, but are preferably substantially linear.

Particularly useful quaternary germicides include compositions which include a single quaternary compound, as well as mixtures of two or more different quaternary compounds. Such useful quaternary compounds are available under the BARDAC®, BARQUAT®, HYAMINE®, LONZABAC®, and ONYXIDE® trademarks, which are more fully described in, for example, McCutcheon's Functional Materials (Vol. 2), North American Edition, 1998, as well as the respective product literature from the suppliers identified below. For example, BARDAC® 205M is described to be a liquid containing alkyl dimethyl benzyl ammonium chloride, octyl decyl dimethyl ammonium chloride; didecyl dimethyl ammonium chloride, and dioctyl dimethyl ammonium chloride (50% active) (also available as 80% active (BARDAC® 208M)); described generally in McCutcheon's as a combination of alkyl dimethyl benzyl ammonium chloride and dialkyl dimethyl ammonium chloride); BARDAC® 2050 is described to be a combination of octyl decyl dimethyl ammonium chloride/didecyl dimethyl ammonium chloride, and dioctyl dimethyl ammonium chloride (50% active) (also available as 80% active (BARDAC® 2080)); BARDAC® 2250 is described to be didecyl dimethyl ammonium chloride (50% active); BARDAC® LF (or BARDAC® LF-80), described as being based on dioctyl dimethyl ammonium chloride (BARQUAT® MB-50, MX-50, OJ-50 (each 50% liquid) and MB-80 or MX-80 (each 80% liquid) are each described as an alkyl dimethyl benzyl ammonium chloride; BARDAC® 4250 and BARQUAT® 4250Z (each 50% active) or BARQUAT® 4280 and BARQUAT 4280Z (each 80% active) are each described as alkyl dimethyl benzyl ammonium chloride/alkyl dimethyl ethyl benzyl ammonium chloride. Also, HYAMINE® 1622, described as diisobutyl phenoxy ethoxy ethyl dimethyl benzyl ammonium chloride (50% solution); HYAMINE® 3500 (50% actives), described as alkyl dimethyl benzyl ammonium chloride (also available as 80% active (HYAMINE® 3500-80)); and HYMAINE® 2389 described as being based on methyldodecylbenzyl ammonium chloride and/or methyldodecylxylene-bis-trimethyl ammonium chloride. (BARDAC®, BARQUAT® and HYAMINE® are presently commercially available from Lonza, Inc., Fairlawn, N.J.). BTC® 50 NF (or BTC® 65 NF) is described to be alkyl dimethyl benzyl ammonium chloride (50% active); BTC® 99 is described as didecyl dimethyl ammonium chloride (50% active); BTC® 776 is described to be myrisalkonium chloride (50% active); BTC® 818 is described as being octyl decyl dimethyl ammonium chloride, didecyl dimethyl ammonium chloride, and dioctyl dimethyl ammonium chloride (50% active) (available also as 80% active (BTC® 818-80%)); BTC® 824 and BTC® 835 are each described as being of alkyl dimethyl benzyl ammonium chloride (each 50% active); BTC® 885 is described as a combination of BTC® 835 and BTC® 818 (50% active) (available also as 80% active (BTC® 888)); BTC® 1010 is described as didecyl dimethyl ammonium chloride (50% active) (also available as 80% active (BTC® 1010-80)); BTC® 2125 (or BTC® 2125 M) is described as alkyl dimethyl benzyl ammonium chloride and alkyl dimethyl ethylbenzyl ammonium chloride (each 50% active) (also available as 80% active (BTC® 2125 80 or BTC® 2125 M)); BTC® 2565 is described as alkyl dimethyl benzyl ammonium chlorides (50% active) (also available as 80% active (BTC® 2568)); BTC® 8248 (or BTC® 8358) is described as alkyl dimethyl benzyl ammonium chloride (80% active) (also available as 90% active (BTC® 8249)); ONYXIDE® 3300 is described as n-alkyl dimethyl benzyl ammonium saccharinate (95% active). (BTC® and ONYXIDE® are presently commercially available from Stepan Company, Northfield, Ill.) Polymeric quaternary ammonium salts based on these monomeric structures are also considered desirable for the present invention. One example is POLYQUAT®, described as being a 2-butenyldimethyl ammonium chloride polymer.

Other cationic surfactants not particularly enumerated here may also find use in conjunction with the compositions of the present invention.

Exemplary amphoteric surfactants include one or more betaine surfactants according to the general formula:

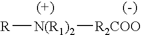

wherein R is a hydrophobic group selected from the group consisting of alkyl groups containing from about 10 to about 22 carbon atoms, preferably from about 12 to about 18 carbon atoms, alkyl aryl and aryl alkyl groups containing a similar number of carbon atoms with a benzene ring being treated as equivalent to about 2 carbon atoms, and similar structures interrupted by amido or ether linkages; each R₁ is an alkyl group containing from 1 to about 3 carbon atoms; and R₂ is an alkylene group containing from 1 to about 6 carbon atoms. Examples of preferred betaines are dodecyl dimethyl betaine, cetyl dimethyl betaine, dodecyl amidopropyldimethyl betaine, tetradecyldimethyl betaine, tetradecylamidopropyldimethyl betaine, and dodecyldimethylammonium hexanoate.

Further exemplary useful amphoteric surfactants include amidoalkylbetaines, such as amidopropylbetaines which may be represented by the following structural formula:

RCONHCH₂CH₂CH₂N⁺(CH₃)₂CH₂COO⁻

wherein R is a straight or branched hydrocarbon chain which may include an aryl moiety, but is preferably a straight hydrocarbon chain containing from about 6 to 30 carbon atoms.

Other amphoteric surfactants not particularly enumerated here may also find use in conjunction with the compositions of the present invention.

Preferably the one or more detersive surfactants include at least one anionic surfactant further with at least one betaine surfactant, especially where one or more cationic surfactants are also present in the inventive compositions. Preferably both anionic and betaine surfactants are present, and the respective weight ratios of the anionic to betaine surfactants is at least about 5:1, more preferably at least about 8:1, still more preferably is at least about 10:1, and most preferably is at least about 12:1.

The detersive surfactants are present in a total amount of 1-35% wt. based on the total weight of the liquid compositions of which they form a part. Preferably however the detersive surfactants are present in amounts of from 2-20% wt. yet more preferably from 5-15% wt.

The inventive compositions necessarily also include 0.01-5% wt. of one or more first biocidal constituent(s) which provide a biocidal effect in conjunction with at least one or more second biocidal constituent(s) selected from benzoic acid, benzoic acid derivatives and/or salts thereof, and farnesol.

The first biocidal constitutent may be one or more antimicrobial agents including: pyrithiones (especially zinc pyrithione which is also known as ZPT), dimethyldimethylol hydantoin (Glydant®), methylchloroisothiazolinone/methylisothiazolinone (Kathon CG®), sodium sulfite, sodium bisulfite, imidazolidinyl urea (Germall 115®), diazolidinyl urea (Germaill II®), benzyl alcohol, 2-bromo-2-nitropropane-1,3-diol (Bronopol®), formalin (formaldehyde), iodopropenyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal dichlorobenzyl alcohol, captan, chlorphenenesin, dichlorophene, chlorbutanol, glyceryl laurate, halogenated diphenyl ethers like 2,4,4′-trichloro-2′-hydroxy-diphenyl ether (Triclosan® or TCS), 2,2′-dihydroxy-5,5′-dibromo-diphenyl ether, phenolic compounds like phenol, 2-methyl phenol, 3-methyl phenol, 4-methyl phenol, 4-ethyl phenol, 2,4-dimethyl phenol, 2,5-dimethyl phenol, 3,4-dimethyl phenol, 2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono- and poly-alkyl and aromatic halophenols such as p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, 5-chloro-2-hydroxydiphenylmethane, resorcinol and its derivatives including methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4′-chloro 2,4-dihydroxydiphenyl methane, 5-bromo 2,4-dihydroxydiphenyl methane, and 4′-bromo 2,4-dihydroxydiphenyl methane, bisphenolic compounds like 2,2′-methylene bis(4-chlorophenol), 2,2′-methylene bis(3,4,6-trichlorophenol), 2,2′-methylene bis(4-chloro-6-bromophenol), bis(2-hydroxy-3,5-dichlorophenyl) sulphide, and bis(2-hydroxy-5-chlorobenzyl)sulphide, benzoic esters (parabens) like methylparaben, propylparaben, butylparaben, ethylparaben, isopropylparaben, isobutylparaben, benzylparaben, sodium methylparaben, and sodium propylparaben, halogenated carbanilides (e.g., 3,4,4′-trichlorocarbanilides (Triclocarban® or TCC), 3-trifluoromethyl-4,4′-dichlorocarbanilide, 3,3′,4-trichlorocarbanilide, etc.). The phenol based non-cationic antimicrobials are preferred, of which para-chloro-meta-xylenol (“PCMX”) is particularly preferred for use in the inventive compositions.

Additionally, one or more cationic surfactants which provide a germicidal or sanitizing benefit may also be used in the first biocidal constituent(s) of the invention. Such cationic surfactants include those described previously with respect to detersive surfactants, and it is to be understood that such cationic surfactants provide dual-functionality to the compositions, namely both a detersive benefit and a biocidal benefit to the inventive compositions.

The second biocidal constituent(s) useful in the inventive compositions are selected from benzoic acid, benzoic acid derivatives and/or salts thereof, and farnesol. By way of non-limiting example, benzoic acid, benzoic acid derivatives and salts thereof useful in the inventive compositions include ortho-hydroxy benzoic acids and derivatives thereof, particularly salicylic acid. Further useful benzoic acid derivatives include the polyhydroxyl carboxylic acids in which at least two hydroxyl groups are present, and at least one of the hydroxyl groups is ortho- to the carboxylic acid group. The remaining hydroxyl group or groups can be in the remaining ortho-, para- or meta-configurations. Preferably the alkyl substituted ortho-hydroxy aromatic carboxylic acids are substituted at the 3, 4 or 5-position, relative to the carboxyl group. Preferred chain lengths for the alkyl group are C₁₋₆, with methyl substituted acids being particularly preferred. Particularly preferred acids are 2-hydroxy 5-methyl benzoic acid, 2-hydroxy 4-methyl benzoic acid and 2-hydroxy 3-methyl benzoic acid.

Farnesol is the common chemical name for 3,7,11-trimethyldodeca-2,6,10-trienol, which is commercially available from a number of sources and has found use in cosmetic compositions, primarily as a fragrance constituent.

As further essential constituents, the inventive compositions include 0.01-7% wt. of one or more pH adjusting constituents. The compositions according to the invention optionally but desirably include an amount of a pH adjusting agent or pH buffer composition. Such compositions include many which are known to the art and which are conventionally used. By way of non-limiting example pH adjusting agents include phosphorus containing compounds, monovalent and polyvalent salts such as of silicates, carbonates, and borates, inorganic and organic acids, bases, tartrates and certain acetates. Exemplary pH adjusting agents include mineral acids, basic compositions, and organic acids, which are typically required in only minor amounts. By way of further non-limiting example pH buffering compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be suitable to use as buffers such materials as aluminosilicates (zeolites), borates, aluminates and certain organic materials such as gluconates, succinates, maleates, and their alkali metal salts. Desirably the compositions according to the invention include an effective amount of an organic acid and/or an inorganic salt form thereof which may be used to adjust and maintain the pH of the compositions of the invention to the desired pH range. Particularly useful is citric acid and metal salts thereof such as sodium citrate, and lactic acid and metal salts thereof, both of which materials which are widely available and which are effective in providing these pH adjustment and buffering effects. These should be screened however to ensure that they do not undesirably complex with or in other ways unduly interfere with the operation of the other constituents present in the inventive compositions.

Desirably the total amount of the pH adjusting agent or pH buffer composition present in the inventive compositions are amount sufficient to establish and maintain the final pH of the compositions to be less than 7, preferably at a pH of 6 or less, preferably a pH of 5 our less, and most preferably at a pH in the range of 4-4.8. Most desirably at least one buffer constituent is present in the inventive compositions; most desirably both citric acid and/or citric acid salts, and lactic acid and/or lactic acid salts are present. While the inventive compositions include 0.01-7% wt. of one or more pH adjusting constituents, preferably these pH adjusting constituents are present in amounts of from 0.01-0.6% wt., yet more preferably from 0.2-0.45% wt., particularly when both citric acid and/or citric acid salts, and lactic acid and/or lactic acid salts are present.

The inventive compositions may comprise 0-10% wt. of one or more organic solvents. By way of non-limiting example exemplary useful organic solvents which may be included in the inventive compositions include those which are at least partially water-miscible such as alcohols (e.g., low molecular weight alcohols, such as, for example, ethanol, propanol, isopropanol, and the like), glycols (such as, for example, ethylene glycol, propylene glycol, hexylene glycol, and the like), water-miscible ethers (e.g. diethylene glycol diethylether, diethylene glycol dimethylether, propylene glycol dimethylether), water-miscible glycol ether (e.g. propylene glycol monomethylether, propylene glycol mono ethylether, propylene glycol monopropylether, propylene glycol monobutylether, ethylene glycol monobutylether, dipropylene glycol monomethylether, diethyleneglycol monobutylether), lower esters of monoalkylethers of ethylene glycol or propylene glycol (e.g. propylene glycol monomethyl ether acetate), and mixtures thereof. Glycol ethers having the general structure Rₐ—R_{b}—OH, wherein Rₐ is an alkoxy of 1 to 20 carbon atoms, or aryloxy of at least 6 carbon atoms, and R_{b} is an ether condensate of propylene glycol and/or ethylene glycol having from one to ten glycol monomer units. Of course, mixtures of two or more organic solvents may be used in the organic solvent constituent.

When present, the organic solvent constituent is desirably present in an amount of from 0.01-5% wt.

According to certain and preferred aspects of the invention, these one or more organic solvents are expressly excluded from the compositions.

The inventive compositions include 0-10% wt. of one or more hydrotropes, particularly one or more hydrotropes based on sulfonated compounds. Organic hydrotropes useful in the use of the compositions of the present invention include known art hydrotrope compositions. Suitable hydrotropes include salts of aryl sulfonic acids such as naphtyl and benzene sulfonic acids, wherein the aromatic nucleus may be unsubstituted or substituted with lower alkyl groups, such as C₁₋₄ alkyl groups, especially methyl, ethyl and/or isopropyl groups. Up to three of such substitutents may be present in the aromatic nucleus, but preferably zero to two are preferred. The salt forming cation of the hydrotrope is preferably an alkali metal such as sodium or potassium, especially sodium. However, other water soluble cations such as ammonium, mono-, di- and tri-lower alkyl, i.e., C₁₋₄ alkanol ammonium groups can be used in the place of the alkali metal cations. Exemplary hydrotropes include benzene sulfonates, o-toluene sulfonates, m-toluene sulfonates, and p-toluene sulfonates; 2,3-xylene sulfonates, 2,4-xylene sulfonates, and 4,6-xylene sulfonates; cumene sulfonates, toluene sulfonates, wherein such exemplary hydrotropes are generally in a salt form thereof, including sodium and potassium salt forms. Further exemplary hydrotropes include lower alkyl sulfate salts, particularly those having from about one to six carbon atoms in the alkyl group.

When present, the hydrotrope constituent is desirably present in an amount of from 0.01-5% wt.

According to certain and preferred aspects of the invention, these one or more hydrotropes are expressly excluded from the compositions.

Further, in certain particularly preferred embodiments both the one or more organic solvents and the one or more hydrotropes are expressly excluded from the inventive compositions. Notwithstanding the absence of these constituents, liquid compositions exhibiting good cleaning and good sanitization are possible, while also exhibiting good storage stability of the liquid compositions, particularly when such are produced in a thickened liquid form as described herein.

The inventors have surprisingly found that storage stable compositions which exclude either the organic solvent constituent, and/or the hydrotrope constituent can be produced which provide both good cleaning and also provide a good biocidal effect to the surfaces treated with such compositions. This is surprising, particularly wherein hydrotrope constituents are omitted from compositions as such are usually required to be present to ensure the compatibility and stability of such compositions. That this has been achieved, particularly also in the further absence of organic compositions is further surprising but nonetheless advantageous to the art.

The compositions of the present invention can also optionally comprise one or more further constituents which are directed to improving the aesthetic or functional features of the inventive compositions. The inventive compositions may include 0-50% wt. of one or more optional constituents including but not limited to: thickeners, viscosity modifiers, coloring agents, opacifying agents, dyes, fragrances, perfumes, preservatives, chelating agents, as well as other optional constituents known to the art although not specifically elucidated here. When one or more of the optional constituents is added, i.e., fragrance and/or coloring agents, the esthetic and consumer appeal of the product is often favorably improved. The use and selection of these optional constituents is well known to those of ordinary skill in the art. When present, the total amount of these one or more optional constituents present in the inventive compositions desirably do not exceed about 20% wt.

According to certain and particularly preferred aspects of the invention, the liquid compositions are provided in a thickened form. Preferably the thickener constituent is present in a sufficient thickening-effective amount such that viscosity of the liquid composition is at least about 2,000 cps, preferably at least about 5,000 cps, yet more preferably the viscosity of the liquid composition is at least about 7,500 cps, and most preferably is in the range of from about 7,500-14,000 cps, as measured using a Brookfield Viscometer Type DV-III, at 12 rpm using spindle #3 at room temperature (approx. 20° C., 68° F.).

Thickeners useful in the present invention to achieve this viscosity are selected from the group consisting of cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, xanthan gum, gellan gum, guar gum and mixtures thereof. Examples of the cellulose derivatives include ethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, and ethyl hydroxy ethyl cellulose. Preferably, the thickener comprises guar gum. Further specific preferred thickeners and combinations of thickeners are described in the Examples. Other thickening materials, particularly those based on synthetic polymers such as acrylic acid copolymers, e.g. Carbopol® materials, may also be included in the inventive compositions. Thickeners based on clays however, are desirably absent from the inventive compositions.

Useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Such chelating agents may be omitted, or they may be included in generally minor amounts such as from 0-0.5% wt. based on the weight of the chelating agents and/or salt forms thereof. Desirably, such chelating agents are included in the present inventive composition in amounts from 0-0.5% wt., but are most desirably present in reduced weight percentages from about 0-0.3% wt.

While the invention compositions may take any visual form, e.g. colored or uncolored, and transparent, translucent or opaque, the use of pearlizers or pearescent materials which are added to both opacify and to provide a sheen to the visual appearance to the liquid compositions of the invention is often desirable. Such materials include known-art materials, and are described in the literature.

The inventive compositions are largely aqueous in nature, and the remaining balance of the compositions, to 100% wt., is provided by water. The water may be tap water, but is preferably distilled and is most preferably deionized water. If the water is tap water, it is preferably substantially free of any undesirable impurities such as organics or inorganics, especially minerals salts which are present in hard water which may thus undesirably interfere with the operation of the constituents present in the aqueous compositions according to the invention.

The compositions of the invention may be packaged in any suitable container, e.g, and can be desirably provided as a ready to use product in a non-pressurized container such as a rigid bottle or a manually squeezable bottle (deformable bottle), as well as in a spray bottle which uses a dip tube and trigger assembly to dispense a liquid, also widely known as a pump-spray apparatus. In use, the user of the inventive composition dispenses a quantity of the composition and applied it to the surface needing treatment, e.g, a dermal or skin surface, hair, or virtually any other surface which may benefit from treatment using the compositions. The inventive compositions are advantageously used as a personal care cleaning product, such as a hand soap or body wash which is primarily intended to clean the skin, and/or a shampoo product which is primarily intended to clean hair. In such applications, an quantity of the composition is provided either directly to the locus of the skin or hair to be treated, and afterwards may be optionally but is desirably rinsed with a further quantity of water.

The inventive compositions may also be provided in a pressurized container, e.g., an aerosol canister containing a quantity of the compositions and a suitable aerosol propellant.

The following examples below illustrate exemplary formulations and preferred formulations of the inventive composition. It is to be understood that these examples are presented by means of illustration only and that further useful formulations fall within the scope of this invention and the claims may be readily produced by one skilled in the art and not deviate from the scope and spirit of the invention. Throughout this specification and in the accompanying claims, weight percents of any constituent are to be understood as the weight percent of the active portion of the referenced constituent, unless otherwise indicated.

### EXAMPLES

An example composition according to the invention was produced by mixing together a the constituents indicated on Table 1 under stirring to produce a homogenous mixture. The individual constituents were “as supplied” from the respective supplier; unless indicated otherwise are to be understood to have been supplied on a “100% wt.” actives basis. The function of the specific constituent is also indicated on Table 1 as well.

TABLE 1

| | Ex. 1 | |
|---|---|---|
| Constituent | % w/w | Function |
| parachlorometaxylenol | 0.175 | first biocidal constituent |
| farnesol | 0.23 | second biocidal |
| | | constituent |
| sodium lauryl ether sulfate (70% wt. | 16 | surfactant |
| actives in aqueous comp.) | | |
| cocoamidopropyl betaine (29% wt. | 2.67 | surfactant |
| actives in aqueous comp.) | | |
| alkyl ether | 1.5 | pearliser |
| sulphate/alkonolamide/alcohol | | |
| ethoxylate | | |
| tetrasodium EDTA (87% wt. actives) | 0.3 | chelating agent |
| sodium lactate | 1 | buffer |
| trisodium citrate dihydrate | 0.7 | buffer |
| Kathon CG | 0.02 | preservative |
| lactic acid | 0.3 | buffer |
| citric acid monohydrate | 0.25 | buffer |
| sodium chloride | 0.6 | viscosity modifier |
| hydroxypropyltriammonium chloride | 0.3 | viscosity modifier |
| perfume (proprietary composition) | 0.2 | fragrance |
| FD&C Blue #1 | 0.00015 | colorant |
| FD&C Green #8 | 0.00468 | colorant |
| deionised water | q.s. | water |

Deionized water was supplied in “quantum sufficient” in order to provide 100% w/w for the composition.

The composition according to Example 1 exhibits good storage stability, and is expected to exhibit good antimicrobial efficacy.

1. A liquid composition for the treatment of the skin and/or hair in order to provide a cleaning and/or sanitizing effect thereto, which comprises:
1-35% wt. of one or more detersive surfactants selected from anionic, nonionic, cationic, and amphoteric surfactants;
0.01-5% wt. of one or more first biocidal constituent(s) which provide a biocidal effect in conjunction with at least one or more second biocidal constituent(s) selected from selected from benzoic acid, benzoic acid derivatives and/or salts thereof, and farnesol;
0.01-7% wt. of one or more pH adjusting constituents;
0-10% wt. of one or more organic solvents;
0-10% wt. of one or more hydrotropes;
0-50% wt. of one or more optional constituents including but not limited to:
thickeners, viscosity modifiers, coloring agents, opacifying agents, dyes, fragrances, perfumes, preservatives, chelating agents; and to 100% wt. water.

2. A liquid composition according to claim 1 which necessarily includes a thickener constituent.

3. A liquid composition according to claim 2 wherein the thickener constituent is present in a thickening-effective amount such that viscosity of the liquid composition is at least about 2,000 cps.

4. A liquid composition according to claim 3 wherein the viscosity of the liquid composition is at least about 5,000 cps.

5. A liquid composition according to claim 1 wherein hydrotropes based on sulfonated compounds are excluded from the compositions.

6. A liquid composition according to claim 1 wherein organic solvents are excluded from the inventive compositions.

7. A liquid composition according to claim 1 wherein both wherein hydrotropes based on sulfonated compounds, and organic solvents are excluded from the inventive compositions.

8. A method for providing a cleaning or sanitizing effect to a surface which comprises the step of:
applying a liquid composition according to claim 1 to a surface an imparting a cleaning or sanitizing effect thereto.

9. A method according to claim 8 wherein the surface is skin or hair.

10. A liquid composition according to claim 1 wherein said first biocidal constituent is parachlorometaxylenol.

11. A liquid composition according to claim 1 wherein said second biocidal constituent is farnesol.
